SEQUENCE LISTING (4 sequences)

SEQ ID NO: 1 - DNA, 24 bp, Artificial Sequence
  Description of Artificial Sequence Synthetic oligonucleotide
aaattaaccccggacttaagggcc

SEQ ID NO: 2 - DNA, 11 bp, Artificial Sequence
  Description of Artificial Sequence Synthetic oligonucleotide
atacgactagc

SEQ ID NO: 3 - DNA, 24 bp, Artificial Sequence
  Description of Artificial Sequence Synthetic primer
agatcaattaatacgatacctgcg

SEQ ID NO: 4 - DNA, 16 bp, Artificial Sequence
  Description of Artificial Sequence Synthetic oligonucleotide
  modified_base (16)..(16): 3′ Inverted dT
cgcaggtatctttttt

(19) United States
(12) Patent Application Publication
(10) Pub. No.: US 2020/0190550 A1
(43) Pub. Date: Jun. 18, 2020

(54) Enzymatic DNA Synthesis Using the Terminal Transferase Activity of Template-Dependent DNA Polymerases

(71) Applicant: President and Fellows of Harvard College, Cambridge, MA (US)

(72) Inventors: Reza Kalhor, East Boston, MA (US); Henry Hung-yi Lee, Brookline, MA (US); George M. Church, Brookline, MA (US)

(21) Appl. No.: 16/615,865

(22) PCT Filed: May 24, 2018
(86) PCT No.: PCT/US18/34365

§ 371 Date: Nov. 22, 2019

Related U.S. Application Data
(60) Provisional application No. 62/510,483, filed on May 24, 2017.

(51) Int. Cl. C12P 19/34; C12N 9/12

(52) U.S. Cl. CPC C12P 19/34; C12Y 207/07031; C12N 9/1264

(57) ABSTRACT

Methods for making a polynucleotide is provided. The methods include (a) providing a first single stranded oligonucleotide, (b) providing a second single stranded oligonucleotide under conditions wherein the first single stranded oligonucleotide anneals to the second single stranded oligonucleotide thereby forming a double stranded oligonucleotide template having an extendible end comprising the 3′ terminal nucleotide of the first single stranded oligonucleotide, (c) providing a reaction mixture to the double stranded initiator wherein the reaction mixture comprises an enzyme, a selected nucleotide triphosphate, and divalent cations, and wherein the enzyme extends the extendible end, (d) regenerating an extendible end of the extended template, and repeating steps (c) to (d) until a polynucleotide of a desired sequence or information content is formed, with the proviso that step (d) is not required to be performed after the polynucleotide is formed.

### RELATED APPLICATION DATA

This application claims priority to U.S. Provisional Application No. 62/510,483 filed on May 24, 2017, which is hereby incorporated herein by reference in its entirety for all purposes.

### STATEMENT OF GOVERNMENT INTERESTS

This invention was made with government support under Grant No. HG005550 and Grant No. MH103910 from the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on May 23, 2018, is named 010498_01105_WO_SL.txt and is 1,341 bytes in size.

### FIELD

The present invention relates in general to methods of making oligonucleotides and polynucleotides using enzymatic synthesis.

### BACKGROUND

DNA synthesis has been a subject of extensive studies in the field of synthetic biology and has broad applications in gene synthesis and information storage. Enzymatic DNA synthesis has recently been proposed as a more effective alternative to chemical DNA synthesis. Unlike DNA replication, de novo synthesis of DNA of a custom sequence requires enzymes that act in a template independent fashion. Since almost all DNA polymerases have evolved for high-fidelity replication of DNA templates, de novo synthesis of custom sequences has been mostly accomplished through chemical synthesis.

Thus far, only one DNA polymerase—Terminal Deoxynucleotidyl Transferase or TdT—is shown to have significant template independent DNA polymerization activity. Given an accessible 3′ hydroxyl group of a DNA strand and available nucleotide triphosphate (dNTP) substrates, this enzyme extends the 3′ end of the DNA strand by subsequent addition of single dNTPs. The enzyme discriminates little among the various dNTPs (i.e., dATP, dCTP, dGTP, dTTP) and thus generally extends the 3′ end of the DNA strand with a random sequence. Despite advances in using TdT for creating DNA strands of a desired sequence or information content, TdT based DNA synthesis still has numerous limitations. For instance, TdT is easily hindered by secondary structure of DNA, has slow kinetics compared to most template dependent DNA polymerases, has very different affinities to different dNTPs. and does not accept many unnatural dNTPs, including almost all 3′-modified varieties. The latter limitation hampers the utility of this enzyme for high-accuracy DNA synthesis with reversible-terminator nucleotide substrates. Together, these limitations make alternative template independent polymerases or template-independent polymerization strategies much desired. There is a continuing need in the art to improve the accuracy, efficiency, and affordability of DNA synthesis.

### SUMMARY

The present disclosure addresses this need and is based on the discovery that certain template-dependent DNA polymerases can synthesize oligonucleotides or polynucleotides of a desired sequence in a template-independent fashion. The methods according to the disclosure use terminal transferase activity of template-dependent DNA polymerases for template-independent DNA synthesis. The disclosure provides novel methods for de novo enzymatic DNA synthesis using the terminal transferase activity of template-dependent DNA polymerases. The disclosure provides for the use of different divalent cations, most importantly manganese, to expand and control the terminal transferase activity of the template-dependent DNA polymerases. The disclosure provides for schemes to carry out terminal transferase-based DNA synthesis with a short cycle time.

Using the terminal transferase activity of template-dependent DNA polymerases to add an adenine residue, or in rarer cases a guanine residue, in a specific fashion to a blunt DNA end has been previously described in the state of art. According to certain aspects, methods are provided where certain cations such as manganese can be used in the enzymatic polymerization reaction to enable template-dependent DNA polymerases to add various dNTPs (e.g., dATP, dCTP, dGTP, dTTP) to the 3′ terminal nucleotide of an extendible end of a double stranded initiator so that an oligonucleotide or polynucleotide of a desired sequence or information content can be synthesized. The extendible end comprises any structure that can be extended by a template-dependent DNA polymerase via its terminal transferase activity. In some embodiments, the extendible end comprises a blunt end, a 5′ overhang, a short 3′ overhang, a mixture thereof, or an equilibrium mixture thereof. The disclosure provides that under ideal circumstances, it is desirable to limit the number of nucleotide additions by the template-dependent DNA polymerases to one. Such DNA is not only suitable for digital information storage but also for use in biological/genetic application. The disclosure further provides that limiting the nucleotide additions to one is not necessarily required for storage of information into DNA. An exemplary proper encoding strategy that, instead of considering each nucleotide base as a unit of information, considers each stretch of one or more identical bases (i.e., a homopolymer) as a unit of information can be used for digital storage purposes. For instance, if every stretch of A or T represents 0 and every stretch of C or G represents 1, the sequence “AAATTAACCCCGGACTTAACGGGCCC” (SEQ ID NO: 1) would be equivalent to “ATACGACTAGC” (SEQ ID NO: 2) and would represent “00011010011”.

According to one aspect, the present invention provides a method for adding one or more selected nucleotides to an extendible end of a double stranded oligonucleotide initiator. The method includes (a) providing a first single stranded oligonucleotide (b) providing a second single stranded oligonucleotide under conditions wherein the first single stranded oligonucleotide anneals to the second single stranded oligonucleotide thereby forming the double stranded oligonucleotide initiator having an extendible end comprising a 3′ terminal nucleotide of the first single stranded oligonucleotide, and (c) providing a reaction mixture to the double stranded initiator wherein the reaction mixture comprises a template-dependent DNA polymerase, one or more selected nucleotide triphosphates, and divalent cations, and wherein the template-dependent DNA polymerase adds one or more of the selected nucleotide triphosphates to the 3′ terminal nucleotide of the first single stranded oligonucleotide of the extendible end of the double stranded oligonucleotide initiator.

According to another aspect, the present invention provides a method for enhancing terminal-transferase activity of a template-dependent polymerase. The method includes supplementing an effective amount of non-magnesium divalent cations to a reaction mixture wherein the reaction mixture comprises i) buffer, salt, and the template-dependent DNA polymerase having terminal-transferase activity, ii) a double stranded oligonucleotide initiator having an extendible end, iii) a selected set of nucleotide triphosphates, and iv) divalent cations, wherein the double stranded oligonucleotide initiator is formed by annealing a first single stranded oligonucleotide to a second single stranded oligonucleotide, and wherein the 3′ terminal nucleotide of the first single stranded oligonucleotide of the extendible end of the double stranded oligonucleotide initiator is extended by the terminal transferase activity of the template-dependent DNA polymerase in a template independent fashion.

In one embodiment, 3′ end terminal nucleotide of the second single stranded oligonucleotide is inactivated from extension. In another embodiment, the 3′ end terminal nucleotide of the second single stranded oligonucleotide lacks a 3′ hydroxyl group for extension. In one embodiment, the extendible end comprises any structure that can be extended by a template-dependent DNA polymerase via its terminal transferase activity. In another embodiment, the extendible end comprises a blunt end, a 5′ overhang, a short 3′ overhang, a mixture thereof, or an equilibrium mixture thereof. In one embodiment, the template-dependent DNA polymerase has terminal transferase activity. In another embodiment, the template-dependent DNA polymerase lacks 3′ to 5′ proofreading activity. In certain embodiments, the template-dependent DNA polymerase comprises Bst. Klenow Exo-, Bsu, Sulfolobus. Taq, Therminator. Deep Vent Exo-. OmniAmp, Vent Exo-, Phi29 Exo-, T4 DNA polymerase Exo-, 17 DNA polymerase Exo-, Tth polymerase, Pfu Exo-, E. coli DNA Polymerase I Exo-, 9°N™ DNA polymerase, Pwo Exo-, Pab Exo-, and the like. In one embodiment, the template-dependent DNA polymerase having terminal transferase activity is mutated or otherwise engineered to have reduced or abrogated dependency on a template. In one embodiment, the nucleotide triphosphate comprises a modified nucleotide analogue, a base-modified non-natural nucleotide analogue, a sugar-modified nucleotide analogue, a triphosphate-modified nucleotide analogue, and/or a natural nucleotide. In some embodiments, the nucleotide triphosphate comprises dATP, dTTP, dCTP, dGTP, or dUTP. In one embodiment, the terminal transferase activity of the template-dependent polymerase is modulated by presence of non-magnesium divalent cations. In another embodiment, the terminal transferase activity of the template-dependent polymerase is modulated by the presence of manganese. In one embodiment, the terminal transferase activity of the template-dependent polymerase is modulated by the presence of cobalt. In another embodiment, the terminal transferase activity of the template-dependent polymerase is modulated by the presence of zinc. In one embodiment, the terminal transferase activity of the template-dependent polymerase is modulated by the presence of nickel. In another embodiment, the terminal transferase activity of the template-dependent polymerase is modulated by presence of non-magnesium divalent cations such that the template-dependent polymerase can add a broadened variety of nucleotide triphosphates to the extendible end. In one embodiment, the terminal transferase activity of the template-dependent polymerase is modulated by presence of non-magnesium divalent cations such that the template-dependent polymerase can add nucleotide triphosphates to the extendible end comprising a blunt end, a 5′ overhang, a short 3′ overhang, a mixture thereof, or an equilibrium mixture thereof with enhanced activity. In one embodiment, the divalent cations comprise magnesium, manganese, cobalt, nickel, zinc, cadmium, or calcium. In another embodiment, the divalent cations comprise one or more of magnesium, manganese, cobalt, nickel, zinc, cadmium, or calcium. In one embodiment, extending is catalyzed by the polymerase which covalently adds one or more selected nucleotide triphosphates to the 3′ terminal nucleotide at the extendible end of the initiator. In one embodiment, only a non-magnesium divalent cation or a mixture of non-magnesium divalent cations is provided in the reaction. In another embodiment, a mixture of magnesium and a non-magnesium divalent cation or non-magnesium divalent cations is provided in the reaction. In one embodiment, the non-magnesium divalent cations comprise cobalt, nickel, zinc, cadmium or calcium. In one embodiment, the non-magnesium divalent cations comprise one or more from but not limited to the group comprising magnesium, manganese, cobalt, nickel, zinc, cadmium, and calcium. In one embodiment, the initiator having the extendible end is immobilized to a support. In another embodiment, the terminal transferase activity of the template-dependent polymerase is modulated by presence of non-magnesium divalent cations. In one embodiment, the terminal transferase activity of the template-dependent polymerase is enhanced by presence of non-magnesium divalent cations. In another embodiment, the terminal transferase activity of the template-dependent polymerase is modulated by presence of non-magnesium divalent cations such that the template-dependent polymerase can add a broadened variety of nucleotide triphosphates to the extendible end. In one embodiment, the terminal transferase activity of the template-dependent polymerase is modulated by presence of non-magnesium divalent cations such that the template-dependent polymerase can add nucleotide triphosphates to the extendible end comprising a blunt end, a 5′ overhang, a short 3′ overhang, a mixture thereof, or an equilibrium mixture thereof with enhanced activity. In one embodiment, the terminal transferase activity of the template-dependent polymerase is enhanced by presence of a non-magnesium divalent cation to increase the number of nucleotides added to the extendible end. In another embodiment, the terminal transferase activity of the template-dependent polymerase is expanded by presence of a non-magnesium divalent cation to include more than one nucleotide triphosphate. In one embodiment, the terminal transferase activity of the template-dependent polymerase is expanded by presence of a non-magnesium divalent cation to include natural and modified nucleotide triphosphates. In another embodiment, the specificity of the terminal transferase activity of the template-dependent polymerase is modulated by the ratio of non-magnesium divalent cations to magnesium. In one embodiment, the specificity of the terminal transferase activity of the template-dependent polymerase is modulated by the ratio of manganese to magnesium. In another embodiment, the specificity of the terminal transferase activity of the template-dependent polymerase is modulated by the ratio of cobalt to magnesium. In one embodiment, the specificity of the terminal transferase activity of the template-dependent polymerase regarding the nucleotide triphosphates is modulated by non-magnesium divalent cations. In another embodiment, the specificity of the terminal transferase activity of the template-dependent polymerase regarding the nucleotide triphosphates is modulated by non-magnesium divalent cations to be made more efficient for a specific nucleotide triphosphate or group of nucleotide triphosphates. In one embodiment, the nucleotide triphosphate to be added by the terminal transferase activity of the template-dependent polymerase regarding the nucleotide triphosphates is selected from a mixture of available nucleotide triphosphates in the reaction by non-magnesium divalent cations. In another embodiment, the specificity of the terminal transferase activity of the template-dependent polymerase is modulated by the ratio of divalent cations

According to one aspect, the present disclosure provides a method for making a polynucleotide comprising (a) providing a first single stranded oligonucleotide, (b) providing a second single stranded oligonucleotide under conditions wherein the first single stranded oligonucleotide anneals to the second single stranded oligonucleotide thereby forming a double stranded oligonucleotide initiator having an extendible end comprising the 3′ terminal nucleotide of the first single stranded oligonucleotide, (c) providing a reaction mixture to the double stranded initiator wherein the reaction mixture comprises a template dependent polymerase with terminal transferase activity, a selected nucleotide triphosphate, and divalent cations, and wherein the polymerase extends the extendible end, (d) regenerating an extendible end of the extended initiator, and (e) repeating steps (c) to (d) until a polynucleotide of a desired sequence or information content is formed, with the proviso that step (d) is not required to be performed after the polynucleotide is formed. In one embodiment, 3′ end terminal nucleotide of the second single stranded oligonucleotide is inactivated from extension. In another embodiment, the 3′ end terminal nucleotide of the second single stranded oligonucleotide lacks an open 3′ hydroxyl group for extension. In one embodiment, regenerating comprises removing the second single stranded oligonucleotide and annealing a new single stranded oligonucleotide to the extended first single stranded oligonucleotide. In some embodiments, the annealed single stranded oligonucleotide is removed physically such as by denaturation, chemical or enzymatic removal or by strand displacement. In one embodiment, 3′ end terminal nucleotide of the new single stranded oligonucleotide is inactivated from extension. In another embodiment, the 3′ end terminal nucleotide of the new single stranded oligonucleotide lacks a 3′ hydroxyl group for extension. In one embodiment, regenerating comprises annealing a new dumbbell adaptor oligonucleotide having a 3′ overhang to the extended initiator, ligating the new dumbbell adaptor oligonucleotide to the extended initiator, and cleaving the dumbbell portion of the adaptor chemically or enzymatically to produce an extendible end. In another embodiment, enzymatic cleavage is carried out by an endonuclease including a restriction enzyme, a CRISPR/Cas endonuclease, a USER enzyme, or the like.

According to another aspect, the present disclosure provides a method for making a polynucleotide including (a) providing a first single stranded oligonucleotide, (b) providing a degenerate or universal single stranded oligonucleotide under conditions wherein the first single stranded oligonucleotide anneals to the degenerate or universal single stranded oligonucleotide thereby forming a double stranded oligonucleotide initiator having an extendible end comprising the 3′ terminal nucleotide of the first single stranded oligonucleotide, (c) providing a reaction mixture to the double stranded initiator wherein the reaction mixture comprises an a template dependent polymerase with terminal transferase activity, a selected nucleotide triphosphate, and divalent cations, and wherein the polymerase extends the extendible end. (d) regenerating an extendible end of the extended initiator, and (e) repeating steps (c) to (d) until a polynucleotide of a desired sequence or information content is formed, with the proviso that step (d) is not required to be performed after the polynucleotide is formed.

In one embodiment, 3′ end terminal nucleotide of the degenerate or universal single stranded oligonucleotide is inactivated from extension. In another embodiment, the 3′ end terminal nucleotide of the degenerate or universal single stranded oligonucleotide lacks a 3′ hydroxyl group for extension. In one embodiment, the extendible end comprises any structure that can be extended by a template-dependent DNA polymerase via its terminal transferase activity. In certain embodiments, the extendible end comprises a blunt end, a 5′ overhang, a short 3′ overhang, a mixture thereof, or an equilibrium mixture thereof. In one embodiment, the universal single stranded oligonucleotide comprises universal bases comprising 3-intropyrrole, 5-nitroindole, or inosine. In certain embodiments, a plurality of degenerate or universal oligonucleotide is provided and excessive degenerate or universal oligonucleotide is removed before extending. In one embodiment, regenerating comprises removing the annealed degenerate or universal single stranded oligonucleotide and annealing a new degenerate or universal single stranded oligonucleotide to the extended first single stranded oligonucleotide. In one embodiment, the annealed degenerate or universal single stranded oligonucleotide is removed by denaturation, chemical or enzymatic removal. In another embodiment, enzymatic removal is carried out by an endonuclease including a restriction enzyme, a CRISPR/Cas endonuclease, a USER enzyme, or the like.

According to yet another aspect of the present disclosure, a method is provided for making a polynucleotide including (a) providing a first single stranded oligonucleotide to a reaction mixture wherein the reaction mixture comprises a degenerate or universal single stranded oligonucleotide attached to an enzyme, a selected nucleotide triphosphate, and divalent cations, (b) subjecting the reaction mixture to a condition wherein the degenerate or universal single stranded oligonucleotide anneals to the first single stranded oligonucleotide thereby forming a double stranded oligonucleotide initiator having an extendible end comprising the 3′ terminal nucleotide of the first single stranded oligonucleotide, (c) extending the extendible end by the enzyme, (d) regenerating an extendible end of the extended template, and (e) repeating steps (c) to (d) until a polynucleotide of a desired sequence or information content is formed, with the proviso that step (d) is not required to be performed after the polynucleotide is formed. In one embodiment, 3′ end terminal nucleotide of the degenerate or universal single stranded oligonucleotide is inactivated from extension. In another embodiment, the 3′ end terminal nucleotide of the degenerate or universal single stranded oligonucleotide lacks a 3′ hydroxyl group for extension. In one embodiment, the extendible end comprises any structure that can be extended by a template-dependent DNA polymerase via its terminal transferase activity. In certain embodiments, the extendible end comprises a blunt end, a 5′ overhang, a short 3′ overhang, a mixture thereof, or an equilibrium mixture thereof. In one embodiment, the enzyme is a template-dependent DNA polymerase. In another embodiment, the template-dependent DNA polymerase has terminal transferase activity. In one embodiment, the template-dependent DNA polymerase lacks 3′ to 5′ proofreading activity. In certain embodiments, the template-dependent DNA polymerase comprises Bst, Klenow Exo-, Bsu, Sulfolobus. Taq, Therminator, Deep Vent Exo-, OmniAmp. Vent Exo-, Phi29 Exo-, T4 DNA polymerase Exo-, T7 DNA polymerase Exo-, Tth polymerase, Pfu Exo-, E. coli DNA Polymerase I Exo-, 9° N™ DNA polymerase. Pwo Exo-. Pab Exo-, and the like. In one embodiment, the template-dependent DNA polymerase having terminal transferase activity is mutated or otherwise engineered to have reduced or abrogated template-dependent polymerization activity. In one embodiment, the nucleotide triphosphate comprises a reversible terminator nucleotide analogue. In another embodiment, the nucleotide triphosphate comprises a modified nucleotide analogue. In one embodiment, the nucleotide triphosphate comprises a base-modified non-natural nucleotide analogue. In another embodiment, the nucleotide triphosphate comprises a sugar-modified nucleotide analogue. In one embodiment, the nucleotide triphosphate comprises a triphosphate-modified nucleotide analogue. In another embodiment, the nucleotide triphosphate comprises a natural nucleotide. In some embodiments, the nucleotide triphosphate comprises dATP, dTTP, dCTP, dGTP, or dUTP. In one embodiment, regenerating comprises removing the degenerate or universal single stranded oligonucleotide attached to the enzyme and annealing a new degenerate or universal single stranded oligonucleotide attached to the enzyme to the extended first single stranded oligonucleotide. In another embodiment, the annealed degenerate or universal single stranded oligonucleotide is removed physically such as by denaturation, chemically, enzymatically or by strand displacement. In one embodiment, enzymatic removal is carried out by an endonuclease including a restriction enzyme, a CRISPR/Cas endonuclease, a USER enzyme, or the like. In one embodiment, extending is catalyzed by the enzyme which covalently adds one or more selected nucleotides to the 3′ terminal nucleotide at the extendible end of the initiator. In one embodiment, the divalent cations comprise magnesium. In another embodiment, the divalent cations comprise manganese. In one embodiment, the divalent cations comprise cobalt. In another embodiment, the divalent cations comprise nickel. In one embodiment, the divalent cations comprise zinc. In one embodiment, the divalent cations comprise cadmium. In another embodiment, the divalent cations comprise calcium. In one embodiment, the first single stranded oligonucleotide is immobilized to a support. In another embodiment, the degenerate or universal single stranded oligonucleotide is attached to the enzyme near its active site. In one embodiment, the active site of the enzyme is modified for increased extension efficiency. In one embodiment, the terminal transferase activity of the template-dependent polymerase is modulated by presence of non-magnesium divalent cations. In another embodiment, the terminal transferase activity of the template-dependent polymerase is enhanced by presence of non-magnesium divalent cations. In one embodiment, the non-magnesium divalent cations comprise cobalt, nickel, zinc, cadmium or calcium. In one embodiment, the non-magnesium divalent cations comprise one or more from but not limited to the group comprising magnesium, manganese, cobalt, nickel, zinc, cadmium, and calcium. In one embodiment, the terminal transferase activity of the template-dependent polymerase is modulated by presence of non-magnesium divalent cations such that the template-dependent polymerase can add a broadened variety of nucleotide triphosphates to the extendible end. In another embodiment, the terminal transferase activity of the template-dependent polymerase is modulated by presence of non-magnesium divalent cations such that the template-dependent polymerase can add nucleotide triphosphates to the extendible end comprising a blunt end, a 5′ overhang, a short 3′ overhang, a mixture thereof, or an equilibrium mixture thereof with enhanced activity. In one embodiment, the terminal transferase activity of the template-dependent polymerase is modulated by the presence of manganese, cobalt, zinc, or nickel.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Further features and advantages of certain embodiments of the present invention will become more fully apparent in the following description of embodiments and drawings thereof, and from the claims.

### BRIEF DESCRIPTION OF THE DRAWINGS

The foregoing and other features and advantages of the present embodiments will be more fully understood from the following detailed description of illustrative embodiments taken in conjunction with the accompanying drawings in which:

FIG. 1 depicts a terminal transferase activity assay. A DNA substrate with a blunt end (bottom left) is prepared by annealing a primer to a shorter complement strand such that the 3′ end of the primer at the blunt end is open while the other 3′ end—that of the complement strand—is chemically blocked. If this substrate is extended at the blunt end by terminal transferase activity of a DNA polymerase, the primer strand extends by one or a few bases, leading to a 3′ overhang (top left). This extension is then detected on a denaturing 15% TBE-Urea gel (right) where the primer and complement strands are separated and migrate to positions in the gel according to their sizes.

FIG. 2 depicts the effect of manganese on the terminal transferase activity of Taq DNA polymerase. Taq DNA polymerase was incubated with the DNA substrate of FIG. 1 and either none or one of dATP, dTTP, dCTP, and dGTP in the absence or presence of manganese. The results of the reaction are resolved on a 15% TBE-Urea gel.

FIGS. 3A-3C depict terminal transferase activity of nine DNA polymerases that have terminal transferase activity with dCTP, with (FIG. 3C) and without (FIG. 3B) manganese in the reaction. FIG. 3A is negative control, lacking dCTP. Each lane corresponds to a different polymerase, 1: Exo- Klenow, 2: Large fragment of Bst, 3: Large fragment of Bsu, 4: Sulfolobus, 5: OmniAmp, 6: Taq, 7: Therminator, 8: Exo- Vent, 9: Exo- Deep-Vent. The right-most lane marked by “L” is the ladder, which is a mixture of the primer and its synthesized variants with one, two, three, four, or five cytosines added to their 3′ end, simulating the products of the extension reaction.

FIG. 4 depicts multiple extension rounds of a primer with degenerate complements through terminal transferase activity. Lane 1: 1 round of thermal cycling. Lane 2: 2 rounds of thermal cycling, Lane 3: 3 rounds of thermal cycling. Lane 4: 5 rounds of thermal cycling. The left-most lane marked by “L” is the ladder, which a mixture of the primer and its synthesized variants with one, two, three, four, or five adenosines added to their 3′ end, simulating the earlier products of the extension reaction.

### DETAILED DESCRIPTION

The present disclosure provides methods for DNA synthesis by using terminal transferase activity of template-dependent DNA polymerases for template-independent DNA synthesis. According to certain embodiments, a first single stranded oligonucleotide is annealed to a second single stranded oligonucleotide to form a double stranded oligonucleotide initiator that has an extendible end. In certain embodiments, the first strand is called a primer or initiator sequence/strand and the second strand is called a complement sequence/strand. In one embodiment, the complement strand is complementary to and shorter than the primer strand so that the annealed double stranded oligonucleotide template has a blunt end at one end and a 5′ overhang of the first strand at the other end. The 3′ recessive terminal nucleotide of the second strand is blocked from extension. In certain embodiments, the extendible end comprises any structure that can be extended by a template-dependent DNA polymerase via its terminal transferase activity in the desired reaction conditions. In some embodiments, the extendible end comprises a blunt end, a 5′ overhang, a short 3′ overhang, a mixture thereof, or an equilibrium mixture thereof. In other embodiments, the extendible end comprises a hybrid between the first strand and another molecule that would mimic a second DNA strand in a manner that enables the terminal transferase activity of a template-dependent polymerase. The 3′ terminal nucleotide at the extendible end of the double stranded oligonucleotide initiator is extended in a reaction mixture comprising a template-dependent DNA polymerase, a selected nucleotide triphosphate, and divalent cations. One or more selected nucleotides can be added to the 3′ terminal nucleotide at the extendible end of the template for each round of polymerization reaction. Since the template-dependent DNA polymerase prefers a double stranded blunt end for addition of selected nucleotides, as the 3′ end grows, the extending 3′ overhang is becoming an increasingly poor substrate for the template-dependent DNA polymerase. The disclosure provides methods and schemes for regenerating an extendible end of the extended double stranded oligonucleotide for each of the subsequent rounds of polymerization until an oligonucleotide or polynucleotide of desired sequence or information content is formed. The disclosure provides methods for modulating the terminal transferase activity of the template-dependent DNA polymerase such as by the use of different divalent cations, most importantly manganese, to expand and control the terminal transferase activity of the template-dependent DNA polymerases so that the initiator can be extended by a desired nucleotide and to a desired length at each round of polymerization reaction. Control of extension time, addition of different selected nucleotides, addition of cations such as one or more of magnesium, manganese, cobalt, nickel, zinc, cadmium or calcium, or deactivation of the template or the enzyme can be used to modulate the addition of nucleotides according to a desired sequence. The present disclosure provides that a different condition can be used for different selected nucleotides. This is important as the kinetics of the enzyme may be different for different selected nucleotides. Thus, to obtain optimal results, different conditions, such as type and concentration of divalent ions may need to be used for different selected nucleotides. In this manner, nucleotide addition can be controlled to a desired number of nucleotides, such as one nucleotide, two nucleotides, three nucleotides etc. The disclosure provides that addition is limited to one nucleotide, two nucleotides, three nucleotides or more during one round of nucleotide addition. This activation or inactivation of the reaction components may be reversible to allow for multiple rounds of nucleotide polymerization that each adds a different nucleotide to the primer or growing polynucleotide chain. The disclosure provides methods for regenerating an extended extendible end template for each round of nucleotide polymerization. The disclosure provides for schemes to carry out terminal transferase-based DNA synthesis with a short cycle time. Additional methods for controlling the nucleotide addition by changing the reaction conditions or components such as by immobilizing the primer/initiator strand to a solid support and using a mobile reagent delivery system have been described in PCT/US 17/24939 hereby incorporated by reference in its entirety.

According to certain embodiments of the present disclosure, the methods involve attaching the primer strand to a solid substrate. In one embodiment, the 5′ end of the primer strand is attached to a solid substrate. In some embodiments, the 3′ end of the complement strand is blocked from extension. In certain embodiments, instead of natural dNTPs, reversible terminator dNTPs can be used. Terminator dNTPs are modified dNTPs that the enzyme can add to a growing DNA primer but cannot extend further. In such a system, after each reversible terminator dNTP extension, the termination is reverted chemically, physically, or enzymatically, followed by the next desired reversible terminator dNTP extension, and so on. In other embodiments, the selected nucleotide is a natural nucleotide or a nucleotide analog.

The present disclosure provides methods of oligonucleotide and polynucleotide synthesis which enable rapid and high-accuracy synthesis of custom DNA sequences by the template-dependent DNA-polymerases. The methods according to the present disclosure can be used for synthesis of cheaper, more accurate and longer custom DNA sequences for various biochemical, biomedical, or biosynthetic applications. Furthermore, given the potential for high-speed DNA synthesis, the methods according to the present disclosure can facilitate the use of DNA as an information storage medium. In this case, a solid-phase synthesis device can be used to record digital information in DNA molecules.

In one embodiment, the reaction mixture includes a buffer comprising a monovalent salt, a divalent salt, a buffering agent, and a reducing agent at a suitable pH and temperature. In another embodiment, the reaction mixture includes a buffer comprising 5 to 200 mM tris-HCl or HEPES, 0.1 to 10 mM manganese chloride or acetate, 0.1 to 50 mM magnesium chloride or acetate, 0.01 to 10.0 mM DTT or B-mercaptoethanol and with a pH of about 2 to 12 and at a temperature of about 10 and 80° C. In another embodiment, the reaction mixture includes a buffer comprising 10 to 20 mM tris-HCl, 2 to 8 mM manganese chloride, 2 to 8 mM magnesium chloride, 0.5 to 1.0 mM DTT and with a pH of about 2 to 12 and at a temperature of about 10 and 80° C. In one embodiment, the reaction mixture includes a buffer comprising 10 mM tris-HCl, 4 mM manganese chloride, 7 mM magnesium chloride, 0.7 mM DTT and with a pH of about 8.0 and at a temperature of about 37° C.

In some embodiments, a primer sequence is attached to a solid support by a cleavable moiety. The method according to the disclosure further comprises releasing the polynucleotide from the reaction mixture after the desired sequence of nucleotides has been added to the 3′ end of the polynucleotide. The method according to the disclosure further comprises releasing the polynucleotide from the reaction mixture using an enzyme, a chemical, light, heat or other suitable method or reagent. The method according to the disclosure further comprises releasing the polynucleotide from the reaction mixture, collecting the polynucleotide, amplifying the polynucleotide and sequencing the polynucleotide.

The term “polymerase,” as used herein, generally refers to any enzyme capable of catalyzing a polymerization reaction, and variants, mutants, or homologues thereof. Examples of polymerases include, without limitation, a DNA or RNA polymerase, a terminal deoxynucleotidyl transferase (TdT), a transcriptase, and variants, mutants, or homologues thereof. A polymerase can be a polymerization enzyme. In certain embodiments, the enzymes capable of catalyzing a polymerization reaction include template-dependent or template-independent polymerases. In certain embodiments, the polymerases include

(3′→5′ exo-) Escherichia coli DNA Polymerase I, Bst polymerase which is Bacillus stearothermophilus DNA Polymerase, Bsu polymerase which is the large fragment Bacillus subtilis DNA polymerase I, Klenow exo- or Klenow Fragment (3′→5′ exo-) which is an N-terminal truncation of Escherichia coli DNA Polymerase I which retains polymerase activity, but has lost the 5′→3′ exonuclease activity and has mutations (D355A, E357A) which abolish the 3′→5′ exonuclease activity, sulfolobus which is DNA polymerase IV from Sulfolobus islandicus, Taq which is the thermostable DNA polymerase from Thermus aquaticus. Therminator DNA Polymerase is a 9° N™ DNA Polymerase with D141A. E143A, and A485L mutations with an enhanced ability to incorporate modified substrates such as dideoxynucleotides, ribonucleotides and acyclonucleotides, 9° N™ DNA polymerase which is the polymerase from Thermococcus species 9° N-7, Deep Vent Exo- which is Deep Vent (D141A/E143A) DNA Polymerase gene, a genetically engineered form of the native DNA polymerase from Pyrococcus species GB-D, OmniAmp, Vent Exo- which is the Vent (D141A/E143A) DNA Polymerase gene, a genetically engineered form of the native DNA polymerase from Thermococcus litoralis. Phi29 exo- which is the phi29 DNA Polymerase gene from bacteriophage phi29 with mutations that eliminate its 3′→5′ exonuclease activity. T4 exo- which is the T4 DNA Polymerase gene from bacteriophage T4 with mutations that eliminate its 3′→5′ exonuclease activity, T7 DNA polymerase Exo- which is the DNA polymerase from bacteriophage T7 with mutations that eliminate its 3′→5′ exonuclease activity, T7 DNA Polymerase which consists T7 gene 5 protein and E. coli thioredoxin. Tth polymerase which is the thermostable DNA polymerase from Thermus thermophilus HB-8, Pfu exo- polymerase which is the thermostable DNA polymerase from Pyrococcus furiosus with mutations that eliminate its 3′→5′ exonuclease activity, Pwo exo- polymerase which is the thermostable DNA polymerase from Pyrococcus woesei with mutations that eliminate its 3′→5′ exonuclease activity, Pab exo- polymerase which is the thermostable DNA polymerase from Pyrococcus abyssiwith mutations that eliminate its 3′→5′ exonuclease activity.

According to certain embodiments, polymerases, including without limitation template-dependent polymerases, modified or otherwise, can be used to create nucleotide polymers having a random or known or desired sequence of nucleotides. Template-dependent polymerases, whether modified or otherwise, can be used to create the nucleic acids de novo. Preferably the template-dependent polymerases lack the 3′ to 5′ exonuclease activity. Ordinary nucleotides are used, such as A, T/U, C or G. Nucleotides may be used which have chain terminating moieties. Reversible terminators may be used in the methods of making the nucleotide polymers.

Oligonucleotide sequences or polynucleotide sequences are synthesized using a template dependent polymerase, and common or natural nucleic acids, which may be unmodified. Nucleotides (“dNTPs”) with blocking groups or reversible terminators can be used with the dNTPs under reaction conditions that are sufficient to limit or reduce the probability of enzymatic addition of the dNTP to one dNTP, i.e. one dNTP is added using the selected reaction conditions taking into consideration the reaction kinetics. Nucleotides with blocking groups or reversible terminators are known to those of skill in the art. According to an additional embodiment when reaction conditions permit, more than one dNTP may be added with a template dependent polymerase.

Terms and symbols of nucleic acid chemistry, biochemistry, genetics, and molecular biology used herein follow those of standard treatises and texts in the field. e.g., Kornberg and Baker. DNA Replication, Second Edition (W.H. Freeman, New York, 1992); Lehninger, Biochemistry, Second Edition (Worth Publishers, New York, 1975); Strachan and Read, Human Molecular Genetics. Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

As used herein, the terms “nucleic acid molecule.” “nucleic acid sequence.” “nucleic acid fragment” and “oligomer” are used interchangeably and are intended to include, but are not limited to, a polymeric form of nucleotides that may have various lengths, including either deoxyribonucleotides or ribonucleotides, or analogs thereof.

In general, the terms “nucleic acid molecule,” “nucleic acid sequence.” “nucleic acid fragment.” “oligonucleotide” and “polynucleotide” are used interchangeably and are intended to include, but not limited to, a polymeric form of nucleotides that may have various lengths, either deoxyribonucleotides (DNA) or ribonucleotides (RNA), or analogs thereof. A oligonucleotide is typically composed of a specific sequence of four nucleotide bases: adenine (A); cytosine (C); guanine (G); and thymine (T) (uracil (U) for thymine (T) when the polynucleotide is RNA). According to certain aspects, deoxynucleotides (dNTPs, such as dATP, dCTP, dGTP, dTTP) may be used. According to certain aspects, ribonucleotide triphosphates (rNTPs) may be used. According to certain aspects, ribonucleotide diphosphates (rNDPs) may be used.

The term “oligonucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Oligonucleotides may optionally include one or more non-standard nucleotide(s), nucleotide analog(s) and/or modified nucleotides. The present disclosure contemplates any deoxyribonucleotide or ribonucleotide and chemical variants thereof, such as methylated, hydroxymethylated or glycosylated forms of the bases, and the like. According to certain aspects, natural nucleotides are used in the methods of making the nucleic acids. Natural nucleotides lack chain terminating moieties.

Examples of modified nucleotides include, but are not limited to diaminopurine, S2T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS).

Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A. Lavergne T, Welte W, Diederichs K, Dwyer T J. Ordoukhanian P. Romesberg F E, Marx A (2012) KlenTaq polymerase replicates unnatural base pairs by inducing a Watson-Crick geometry, Nature Chem. Biol. 8:612-614; See Y J, Malyshev D A. Lavergne T. Ordoukhanian P, Romesberg F E. J Am Chem Soc. 2011 Dec. 14; 133(49):19878-88, Site-specific labeling of DNA and RNA using an efficiently replicated and transcribed class of unnatural base pairs; Switzer C Y, Moroney S E, Benner S A. (1993) Biochemistry, 32(39):10489-96. Enzymatic recognition of the base pair between isocytidine and isoguanosine; Yamashige R. Kimoto M. Takezawa Y, Sato A, Mitsui T, Yokoyama S. Hirao I. Nucleic Acids Res. 2012 March; 40(6):2793-806. Highly specific unnatural base pair systems as a third base pair for PCR amplification; and Yang Z, Chen F, Alvarado J B, Benner S A. J Am Chem Soc. 2011 Sep. 28; 133(38):15105-12, Amplification, mutation, and sequencing of a six-letter synthetic genetic system. Other non-standard nucleotides may be used such as described in Malyshev, D. A., et al., Nature, vol. 509. pp. 385-388 (15 May 2014) hereby incorporated by reference in its entirety.

Tags of the disclosure may be atoms or molecules, or a collection of atoms or molecules. A tag may provide an optical, electrochemical, magnetic, or electrostatic (e.g., inductive, capacitive) signature, which signature may be detected during the incorporation of nucleotides. A nucleotide can include a tag (or tag species) that is coupled to any location of the nucleotide including, but not limited to a phosphate (e.g., gamma phosphate), sugar or nitrogenous base moiety of the nucleotide. In some cases, tags are detected while tags are associated with a polymerase during the incorporation of nucleotide tags.

In certain exemplary embodiments, one or more oligonucleotide sequences described herein are immobilized on a support (e.g., a solid and/or semi-solid support). In certain aspects, an oligonucleotide sequence can be attached to a support using one or more of the phosphoramidite linkers described herein. Suitable supports include, but are not limited to, slides, beads, chips, particles, strands, gels, sheets, tubing, spheres, containers, capillaries, pads, slices, films, plates and the like. In various embodiments, a solid support may be biological, nonbiological, organic, inorganic, or any combination thereof. Supports of the present invention can be any shape, size, or geometry as desired. For example, the support may be square, rectangular, round, flat, planar, circular, tubular, spherical, and the like. When using a support that is substantially planar, the support may be physically separated into regions, for example, with trenches, grooves, wells, or chemical barriers (e.g., hydrophobic coatings, etc.). Supports may be made from glass (silicon dioxide), metal, ceramic, polymer or other materials known to those of skill in the art. Supports may be a solid, semi-solid, elastomer or gel. In certain exemplary embodiments, a support is a microarray. As used herein, the term “microarray” refers in one embodiment to a type of array that comprises a solid phase support having a substantially planar surface on which there is an array of spatially defined non-overlapping regions or sites that each contain an immobilized hybridization probe. “Substantially planar” means that features or objects of interest, such as probe sites, on a surface may occupy a volume that extends above or below a surface and whose dimensions are small relative to the dimensions of the surface. For example, beads disposed on the face of a fiber optic bundle create a substantially planar surface of probe sites, or oligonucleotides disposed or synthesized on a porous planar substrate create a substantially planar surface. Spatially defined sites may additionally be “addressable” in that its location and the identity of the immobilized probe at that location are known or determinable.

The solid supports can also include a semi-solid support such as a compressible matrix with both a solid and a liquid component, wherein the liquid occupies pores, spaces or other interstices between the solid matrix elements. Preferably, the semi-solid support materials include polyacrylamide, cellulose, poly dimethyl siloxane, polyamide (nylon) and cross-linked agarose, -dextran and -polyethylene glycol. Solid supports and semi-solid supports can be used together or independent of each other.

Supports can also include immobilizing media. Such immobilizing media that are of use according to the invention are physically stable and chemically inert under the conditions required for nucleic acid molecule deposition and amplification. A useful support matrix withstands the rapid changes in, and extremes of, temperature required for PCR. The support material permits enzymatic nucleic acid synthesis. If it is unknown whether a given substance will do so, it is tested empirically prior to any attempt at production of a set of arrays according to the invention. According to one embodiment of the present invention, the support structure comprises a semi-solid (i.e., gelatinous) lattice or matrix, wherein the interstices or pores between lattice or matrix elements are filled with an aqueous or other liquid medium; typical pore (or ‘sieve’) sizes are in the range of 100 μm to 5 nm. Larger spaces between matrix elements are within tolerance limits, but the potential for diffusion of amplified products prior to their immobilization is increased. The semi-solid support is compressible. The support is prepared such that it is planar, or effectively so, for the purposes of printing. For example, an effectively planar support might be cylindrical, such that the nucleic acids of the array are distributed over its outer surface in order to contact other supports, which are either planar or cylindrical, by rolling one over the other. Lastly, a support material of use according to the invention permits immobilizing (covalent linking) of nucleic acid features of an array to it by means known to those skilled in the art. Materials that satisfy these requirements comprise both organic and inorganic substances, and include, but are not limited to, polyacrylamide, cellulose and polyamide (nylon), as well as cross-linked agarose, dextran or polyethylene glycol.

One embodiment is directed to a thin polyacrylamide gel on a glass support, such as a plate, slide or chip. A polyacrylamide sheet of this type is synthesized as follows. Acrylamide and bis-acrylamide are mixed in a ratio that is designed to yield the degree of crosslinking between individual polymer strands (for example, a ratio of 38:2 is typical of sequencing gels) that results in the desired pore size when the overall percentage of the mixture used in the gel is adjusted to give the polyacrylamide sheet its required tensile properties. Polyacrylamide gel casting methods are well known in the art (see Sambrook et al., 1989, Molecular Cloning. A Laboratory Manual, 2nd Edition, Cold Spring Harbor Laboratory Press, Cold Spring Harbor, N.Y., incorporated herein in its entirety by reference), and one of skill has no difficulty in making such adjustments.

The gel sheet is cast between two rigid surfaces, at least one of which is the glass to which it will remain attached after removal of the other. The casting surface that is to be removed after polymerization is complete is coated with a lubricant that will not inhibit gel polymerization; for this purpose, silane is commonly employed. A layer of silane is spread upon the surface under a fume hood and allowed to stand until nearly dry. Excess silane is then removed (wiped or, in the case of small objects, rinsed extensively) with ethanol. The glass surface which will remain in association with the gel sheet is treated with γ-methaciyloxypropyltrimethoxysilane (Cat. No. M6514, Sigma; St. Louis, Mo.), often referred to as ‘crosslink silane’, prior to casting. The glass surface that will contact the gel is triply-coated with this agent. Each treatment of an area equal to 1200 cm² requires 125 μl of crosslink silane in 25 ml of ethanol. Immediately before this solution is spread over the glass surface, it is combined with a mixture of 750 μl water and 75 μl glacial acetic acid and shaken vigorously. The ethanol solvent is allowed to evaporate between coatings (about 5 minutes under a fume hood) and, after the last coat has dried, excess crosslink silane is removed as completely as possible via extensive ethanol washes in order to prevent ‘sandwiching’ of the other support plate onto the gel. The plates are then assembled and the gel cast as desired.

The only operative constraint that determines the size of a gel that is of use according to the invention is the physical ability of one of skill in the art to cast such a gel. The casting of gels of up to one meter in length is, while cumbersome, a procedure well known to workers skilled in nucleic acid sequencing technology. A larger gel, if produced, is also of use according to the invention. An extremely small gel is cut from a larger whole after polymerization is complete.

Note that at least one procedure for casting a polyacrylamide gel with bioactive substances, such as enzymes, entrapped within its matrix is known in the art (O'Driscoll, 1976. Methods Enzymol., 44: 169-183, incorporated herein in its entirety by reference). A similar protocol, using photo-crosslinkable polyethylene glycol resins, that permit entrapment of living cells in a gel matrix has also been documented (Nojima and Yamada, 1987, Methods Enzymol., 136: 380-394, incorporated herein in its entirety by reference). Such methods are of use according to the invention. As mentioned below, whole cells are typically cast into agarose for the purpose of delivering intact chromosomal DNA into a matrix suitable for pulsed-field gel electrophoresis or to serve as a “lawn” of host cells that will support bacteriophage growth prior to the lifting of plaques according to the method of Benton and Davis (see Maniatis et al., 1982, Molecular Cloning: A Laboratory Manual, Cold Spring Harbor Laboratory Press, Cold Spring Harbor. NY. incorporated herein in its entirety by reference). In short, electrophoresis-grade agarose (e.g., Ultrapure; Life Technologies/Gibco-BRL) is dissolved in a physiological (isotonic) buffer and allowed to equilibrate to a temperature of 50° C. to 52° C. in a tube, bottle or flask. Cells are then added to the agarose and mixed thoroughly, but rapidly (if in a bottle or tube, by capping and inversion, if in a flask, by swirling), before the mixture is decanted or pipetted into a gel tray. If low-melting point agarose is used, it may be brought to a much lower temperature (down to approximately room temperature, depending upon the concentration of the agarose) prior to the addition of cells. This is desirable for some cell types; however, if electrophoresis is to follow cell lysis prior to covalent attachment of the molecules of the resultant nucleic acid pool to the support, it is performed under refrigeration, such as in a 4° C. to 10° C. ‘cold’ room.

Oligonucleotides immobilized on microarrays include nucleic acids that are generated in or from an assay reaction. Typically, the oligonucleotides or polynucleotides on microarrays are single stranded and are covalently attached to the solid phase support, usually by a 5′-end or a 3′-end. In certain exemplary embodiments, probes are immobilized via one or more cleavable linkers. The density of non-overlapping regions containing nucleic acids in a microarray is typically greater than 100 per cm², and more typically, greater than 1000 per cm². Microarray technology relating to nucleic acid probes is reviewed in the following exemplary references; Schena, Editor, Microarrays: A Practical Approach (IRL Press, Oxford, 2000); Southern, Current Opin. Chem. Biol., 2: 404-410 (1998); Nature Genetics Supplement, 21:1-60 (1999); and Fodor et al, U.S. Pat. Nos. 5,424,186; 5,445,934; and 5,744,305.

Methods of immobilizing oligonucleotides to a support are known in the art (beads: Dressman et al. (2003) Proc. Natl. Acad. Sci. USA 100:8817, Brenner et al. (2000) Nat. Biotech, 18:630, Albretsen et al. (1990) Anal. Biochem. 189:40, and Lang et al. Nucleic Acids Res. (1988) 16:10861; nitrocellulose: Ranki et al. (1983) Gene 21:77; cellulose: Goldkorn (1986) Nucleic Acids Res. 14:9171; polystyrene: Ruth et al. (1987) Conference of Therapeutic and Diagnostic Applications of Synthetic Nucleic Acids, Cambridge U.K.; teflon-acrylamide; Duncan et al. (1988) Anal. Biochem. 169:104; polypropylene: Polsky-Cynkin et al. (1985) Clin. Chem. 31:1438; nylon: Van Ness et al. (1991) Nucleic Acids Res. 19:3345; agarose: Polsky-Cynkin et al., Clin. Chem. (1985) 31:1438; and sephacryl: Langdale et al. (1985) Gene 36:201; latex: Wolf et al. (1987) Nucleic Acids Res. 15:2911). Supports may be coated with attachment chemistry or polymers, such as amino-silane, NHS-esters, click chemistry, polylysine, etc., to bind a nucleic acid to the support.

As used herein, the term “attach” refers to both covalent interactions and noncovalent interactions. A covalent interaction is a chemical linkage between two atoms or radicals formed by the sharing of a pair of electrons (i.e., a single bond), two pairs of electrons (i.e., a double bond) or three pairs of electrons (i.e., a triple bond). Covalent interactions are also known in the art as electron pair interactions or electron pair bonds. Noncovalent interactions include, but are not limited to, van der Waals interactions, hydrogen bonds, weak chemical bonds (i.e., via short-range noncovalent forces), hydrophobic interactions, ionic bonds and the like. A review of noncovalent interactions can be found in Alberts et al., in Molecular Biology of the Cell, 3d edition, Garland Publishing, 1994.

According to certain aspects, affixing or immobilizing nucleic acid molecules to the substrate is performed using a covalent linker that is selected from the group that includes oxidized 3-methyl uridine, an acrylyl group and hexaethylene glycol. In addition to the attachment of linker sequences to the molecules of the pool for use in directional attachment to the support, a restriction site or regulatory element (such as a promoter element, cap site or translational termination signal), is, if desired, joined with the members of the pool. Nucleic acids that have been synthesized on the surface of a support may be removed, such as by a cleavable linker or linkers known to those of skill in the art. Linkers can be designed with chemically reactive segments which are optionally cleavable with agents such as enzymes, light, heat, pH buffers, and redox reagents. Such linkers can be employed to pre-fabricate an in situ solid-phase inactive reservoir of a different solution-phase primer for each discrete feature. Upon linker cleavage, the primer would be released into solution for PCR, perhaps by using the heat from the thermocycling process as the trigger.

It is also contemplated that affixing of nucleic acid molecules to the support is performed via hybridization of the members of the pool to nucleic acid molecules that are covalently bound to the support.

Immobilization of nucleic acid molecules to the support matrix according to the invention is accomplished by any of several procedures. Direct immobilizing via the use of 3′-terminal tags bearing chemical groups suitable for covalent linkage to the support, hybridization of single-stranded molecules of the pool of nucleic acid molecules to oligonucleotide primers already bound to the support, or the spreading of the nucleic acid molecules on the support accompanied by the introduction of primers, added either before or after plating, that may be covalently linked to the support, may be performed. Where pre-immobilized primers are used, they are designed to capture a broad spectrum of sequence motifs (for example, all possible multimers of a given chain length, e.g., hexamers), nucleic acids with homology to a specific sequence or nucleic acids containing variations on a particular sequence motif. Alternatively, the primers encompass a synthetic molecular feature common to all members of the pool of nucleic acid molecules, such as a linker sequence.

Two means of crosslinking a nucleic acid molecule to a polyacrylamide gel sheet will be discussed in some detail. The first (provided by Khrapko et al., 1996, U.S. Pat. No. 5,552,270) involves the 3′ capping of nucleic acid molecules with 3-methyl uridine. Using this method, the nucleic acid molecules of the libraries of the present invention are prepared so as to include this modified base at their 3′ ends. In the cited protocol, an 8% polyacrylamide gel (30:1, acrylamide: bis-acrylamide) sheet 30 μm in thickness is cast and then exposed to 50% hydrazine at room temperature for 1 hour. Such a gel is also of use according to the present invention. The matrix is then air dried to the extent that it will absorb a solution containing nucleic acid molecules, as described below. Nucleic acid molecules containing 3-methyl uridine at their 3′ ends are oxidized with 1 mM sodium periodate (NaIO₄) for 10 minutes to 1 hour at room temperature, precipitated with 8 to 10 volumes of 2% LiClO₄ in acetone and dissolved in water at a concentration of 10 pmol/μl. This concentration is adjusted so that when the nucleic acid molecules are spread upon the support in a volume that covers its surface evenly and is efficiently (i.e., completely) absorbed by it, the density of nucleic acid molecules of the array falls within the range discussed above. The nucleic acid molecules are spread over the gel surface and the plates are placed in a humidified chamber for 4 hours. They are then dried for 0.5 hour at room temperature and washed in a buffer that is appropriate to their subsequent use. Alternatively, the gels are rinsed in water, re-dried and stored at −20° C. until needed. It is thought that the overall yield of nucleic acid that is bound to the gel is 80% and that of these molecules, 98% are specifically linked through their oxidized 3′ groups.

A second crosslinking moiety that is of use in attaching nucleic acid molecules covalently to a polyacrylamide sheet is a 5′ acrylyl group, which is attached to the primers. Oligonucleotide primers bearing such a modified base at their 5′ ends may be used according to the invention. In particular, such oligonucleotides are cast directly into the gel, such that the acrylyl group becomes an integral, covalently bonded part of the polymerizing matrix. The 3′ end of the primer remains unbound, so that it is free to interact with, and hybridize to, a nucleic acid molecule of the pool and prime its enzymatic second-strand synthesis.

Alternatively, hexaethylene glycol is used to covalently link nucleic acid molecules to nylon or other support matrices (Adams and Kron, 1994. U.S. Pat. No. 5,641,658). In addition, nucleic acid molecules are crosslinked to nylon via irradiation with ultraviolet light. While the length of time for which a support is irradiated as well as the optimal distance from the ultraviolet source is calibrated with each instrument used due to variations in wavelength and transmission strength, at least one irradiation device designed specifically for crosslinking of nucleic acid molecules to hybridization membranes is commercially available (Stratalinker, Stratagene). It should be noted that in the process of crosslinking via irradiation, limited nicking of nucleic acid strands occurs. The amount of nicking is generally negligible, however, under conditions such as those used in hybridization procedures. In some instances, however, the method of ultraviolet crosslinking of nucleic acid molecules will be unsuitable due to nicking. Attachment of nucleic acid molecules to the support at positions that are neither 5′- nor 3′-terminal also occurs, but it should be noted that the potential for utility of an array so crosslinked is largely uncompromised, as such crosslinking does not inhibit hybridization of oligonucleotide primers to the immobilized molecule where it is bonded to the support.

Supports described herein may have one or more optically addressable virtual electrodes associated therewith such that an anion toroidal vortex can be created at a reaction site on the supports described herein.

According to certain aspects, reagents and washes are delivered that the reactants are present at a desired location for a desired period of time to, for example, covalently attached dNTP to an initiator sequence or an existing nucleotide attached at the desired location. A selected nucleotide reagent liquid is pulsed or flowed or deposited at the reaction site where reaction takes place and then may be optionally followed by delivery of a buffer or wash that does not include the nucleotide. Suitable delivery systems include fluidics systems, microfluidics systems, syringe systems, ink jet systems, pipette systems and other fluid delivery systems known to those of skill in the art. Various flow cell embodiments or flow channel embodiments or microfluidic channel embodiments are envisioned which can deliver separate reagents or a mixture of reagents or washes using pumps or electrodes or other methods known to those of skill in the art of moving fluids through channels or microfluidic channels through one or more channels to a reaction region or vessel where the surface of the substrate is positioned so that the reagents can contact the desired location where a nucleotide is to be added. According to another embodiment, a microfluidic device is provided with one or more reservoirs which include one or more reagents which are then transferred via microchannels to a reaction zone where the reagents are mixed and the reaction occurs. Such microfluidic devices and the methods of moving fluid reagents through such microfluidic devices are known to those of skill in the art.

Immobilized nucleic acid molecules may, if desired, be produced using a device (e.g., any commercially-available inkjet printer, which may be used in substantially unmodified form) which sprays a focused burst of reagent-containing solution onto a support (see Castellino (1997) Genome Res. 7:943-976, incorporated herein in its entirety by reference). Such a method is currently in practice at Incyte Pharmaceuticals and Rosetta Biosystems, Inc., the latter of which employs “minimally modified Epson inkjet cartridges” (Epson America. Inc.; Torrance, Calif.). The method of inkjet deposition depends upon the piezoelectric effect, whereby a narrow tube containing a liquid of interest (in this case, oligonucleotide synthesis reagents) is encircled by an adapter. An electric charge sent across the adapter causes the adapter to expand at a different rate than the tube, and forces a small drop of liquid reagents from the tube onto a coated slide or other support.

Reagents can be deposited onto a discrete region of the support, such that each region forms a feature of the array. The feature is capable of generating an anion toroidal vortex as described herein. The desired nucleic acid sequence can be synthesized drop-by-drop at each position, as is true for other methods known in the art. If the angle of dispersion of reagents is narrow, it is possible to create an array comprising many features. Alternatively, if the spraying device is more broadly focused, such that it disperses nucleic acid synthesis reagents in a wider angle, as much as an entire support is covered each time, and an array is produced in which each member has the same sequence (i.e., the array has only a single feature).

### Template-Dependent Polymerases Catalyze 3′ Terminal Nucleotide Addition of a Selected Nucleotide

A limited and specific template-independent polymerization activity in some standard template-dependent DNA polymerases has been long described (Clark 1988). This activity, generally known as terminal transferase activity, involves adding a single or two dATPs to the 3′ end of a blunt double-stranded DNA fragment by template-dependent DNA polymerases that lack proof-reading 3′ to 5′ exonuclease activity (Clark et al. 1987; Yang 2002). Examples of such polymerases include Taq DNA polymerase (Mole et al. 1989; Clark 1988) and Exo-minus fragment of Klenow (Derbyshire et al. 1988). The application of this behavior, which is a technique known as A-tailing, has widespread use in cloning and library preparation. More recently, a specific polymerase that shows this tailing activity with dGTP instead of dATP has been described (Mead et al. n.d.). This limited terminal transferase activity has limited utility for de novo DNA synthesis because it cannot add all four nucleotide types (i.e., A, C, G, and T). Using this activity for de novo DNA synthesis would ideally allow the addition of any of the four nucleotides, just like TdT would.

It has been noted that various template-dependent DNA polymerases have an inherent affinity to one of the four nucleotides which they are most likely to add when replicating across abasic sites. This preferred nucleotide often tends to be adenine, a.k.a., the “A” rule (Strauss 2002). It has been further noted that the nucleotide specificities of various DNA polymerases can be modulated by introducing non-magnesium divalent metal cations such as manganese, cobalt, cadmium, nickel, calcium, zinc, or others (Miyaki et al. 1977). For example, the template-independent polymerization activity of TdT is particularly affected by divalent cations and can be enhanced or limited for a certain nucleotide based on the presence of a certain divalent cation (Miyaki et al. 1977; Deng & Wu 1981; Delarue et al. 2002; Chang & Bollum 1986; Motea & Berdis 2010).

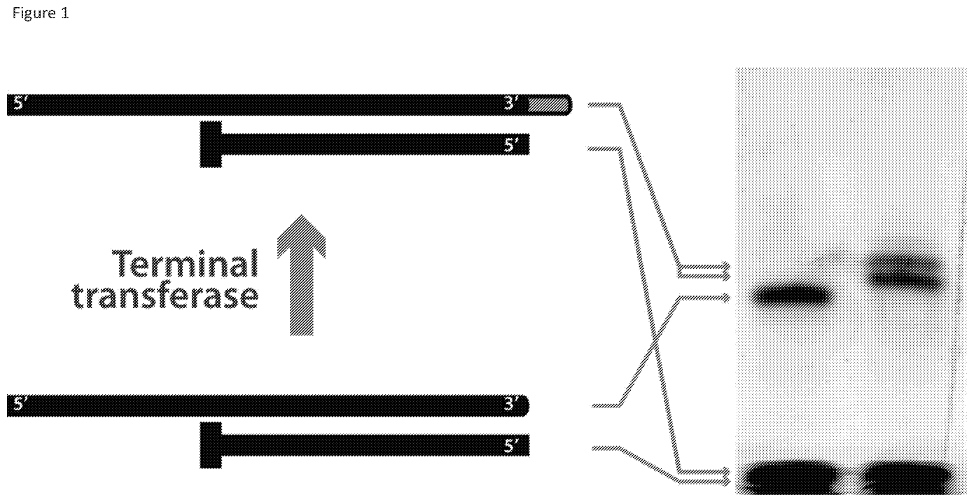

The present disclosure therefore contemplates modulating/enhancing the terminal transferase activity of template-dependent polymerases that lack proofreading activity by altering reaction conditions with non-magnesium divalent cations such that the template-dependent polymerases can accommodate additional nucleotides beyond adenosine and guanosine. In one embodiment, the present disclosure provides a terminal transferase activity assay (FIG. 1). In this assay, polymerization was assessed on a blunt double-stranded DNA end template when incubated with a template-dependent DNA polymerase in presence of a selected dNTP. After a few minutes of incubation, the DNA template is denatured and visualized on a 15% TBE-Urea PAGE gel. Due to the high resolution of the gel, even single nucleotide additions to the blunt end of the dsDNA can be detected and quantified.

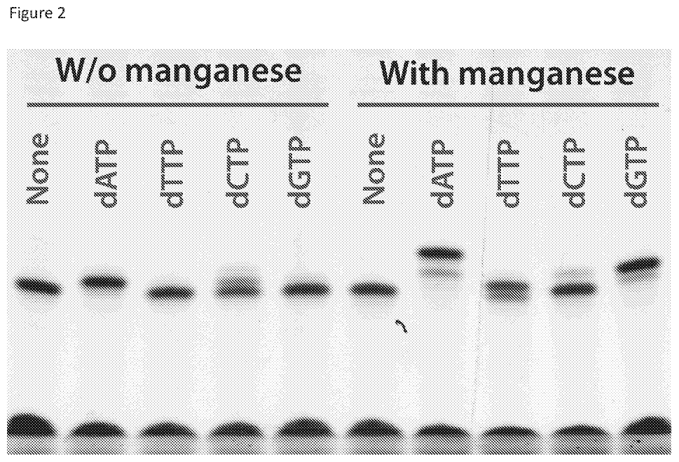

Preliminary analysis using this assay indicated that manganese (Mn) is the most effective non-magnesium divalent metal cation for broadening the terminal transferase activity of Taq polymerase for all dNTPs. In fact, addition of manganese broadened the terminal transferase activity of Taq from only adenosine, to guanosine and cytosine as well with limited use of thymidine (FIG. 2). The effect of manganese on multiple DNA polymerases was then assayed with each of the four nucleotides (i.e., A, C, G. T) to determine the substrate range of their terminal transferase activity. The polymerases tested were Exo-minus Klenow, Bst. Bsu, Sulfolobus DNA Polymerase IV, OmniAmp, Taq, Therminator, Exo-minus Vent, Exo-minus DeepVent which are from a variety of polymerase families and all lack 3′ to 5′ proofreading activity. Tables below show their measured terminal transferase activities for each nucleotide with and without added manganese:

| M− | Exo-Klenow | Bst | Bsu | Sulfolobus | Omni Amp | Taq | Therminator | Vent Exo- | DeepVentExo- |
|---|---|---|---|---|---|---|---|---|---|
| A | +++ | +/− | +/− | − | +/− | +/− | +++ | +/− | − |
| C | +/− | − | − | − | − | − | + | − | − |
| G | + | − | − | − | − | − | ++ | − | − |
| T | +/− | − | − | − | − | − | + | − | − |

| Mn+ | Exo-Klenow | Bst | Bsu | Sulfolobus | Omni Amp | Taq | Therminator | Vent Exo- | DeepVentExo- |
|---|---|---|---|---|---|---|---|---|---|
| A | +++ | +++ | +++ | +/− | +++ | +++ | +++ | +++ | ++ |
| C | +++ | +/− | +/− | +/− | + | +/− | +++ | + | +/− |
| G | +++ | ++ | + | − | +++ | +++ | +++ | ++ | + |
| T | +++ | +/− | +/− | +/− | ++ | + | +++ | + | +/− |

+++) Extension of the entire substrate
++) Extension of more than 50% of the substrate
+) Extension of 10-49% of the substrate
+/−) Extension of less than 10% of the substrate
−) no extension of the substrate

These results show control over the terminal transferase activity of template dependent polymerases by altering the divalent cations in the reaction. More specifically, the use of manganese, a non-physiological divalent cation, expands the substrate specificity and elevates template-independent terminal transferase activity for all assayed DNA polymerases. Furthermore, the results point to Exo-minus Klenow and Therminator as the best candidates for de novo DNA synthesis; they both efficiently add all four nucleotides to a blunt DNA end in the presence of manganese. A skilled in the art can optimize and adjust the reaction condition for optimum result.

### Generalization to De Novo DNA Synthesis

In order to use the biochemistry described above for de novo synthesis of long strands of DNA with an exact sequence or the desired information content, methods of “regenerating the blunt end” have to be implemented. The challenge lies in the fact that a double-stranded DNA terminus is required for terminal transferase activity. Once nucleotides are added to the blunt end, it is converted to a 3′ overhang and this overhang, which is essentially equivalent to single-stranded DNA for the enzyme active site, is no longer a substrate for a template dependent DNA polymerase. In other words, an addition of a nucleotide to the blunt end of the DNA substrate creates a 3′ overhang with subsequent additions making this overhang longer and longer (FIG. 2). The growing overhang is an increasingly poor substrate for the terminal transferase activity of the polymerase, eventually leading to the activity halting altogether (FIG. 2). In order to synthesize longer sequences, it is necessary to actively reconstitute the blunt end as the 3′ end gets extended (regenerating the blunt end), or to create conditions in which even a single-stranded DNA can interact with the enzyme active site similar to how double-stranded DNA would. For instance, a short non-DNA polymer that binds single-stranded initiator near its 3′ end and creates a structure resembling that of double stranded DNA end can be used in the reaction or fused to the DNA polymerase itself. As another instance, the polymerase can be modified and mutated to have a standard or non-standard amino acid at a position in the active site that would occupy the same position as the complementary strand with respect to the primer strand and triggers the terminal transferase activity of the polymerase.

The present disclosure contemplates non-limiting ways of “regenerating the blunt end” to enable synthesis of long strands of DNA. The disclosure methods require regenerating the blunt end after each round of nucleotide addition by the template-dependent DNA polymerase. In one embodiment, regenerating the blunt end can be accomplished by the following steps.

1. Immobilize the 5′ end of a known primer strand on a surface,
2. Anneal a “complement” oligo to the primer in order to generate a blunt end at the 3′ end of the primer.
3. Extend the primer with the desired nucleotide thereby creating a 3′ overhang,
4. Remove the complement oligo by denaturation and washing.
5. Regenerate the blunt end by annealing a new complement oligo based on the extended sequence of the primer,

Steps 3 to 5 can then be repeated in every round of extension until an oligo of a desired sequence is obtained.

In another embodiment, regenerating the blunt end can be accomplished by using a “complement” oligo which is a dumb-bell adapter which enables sticky-end ligation onto the extended 3′ overhang. The dumb-bell is then cleaved chemically, with a restriction enzyme, or with CRISPR, thereby regenerating a blunt end (Mir et al. 2009). For synthesis of biological-grade DNA, the cleavage site would be designed to such that the ligated dumb-bell is “scar-less” for the next base to be extended. For synthesis of information-grade, the cleavage site could be designed with more liberal requirements since leaving a small stretch of DNA bases of known sequence before each extended base could be tolerated—these small stretches could be used to denote the start and end of the bases carrying information and be filtered out in silico.

In other embodiments, regenerating the extendible end can be accomplished by using degenerate complementary oligos. For example, short complementary oligos with a random or degenerate sequence, such as NNNNNN, can be included in the reaction at a high concentration. Similarly, a complementary oligo with a “universal” or “degenerate” nucleotide sequence (Liang et al. 2013; Loakes 2001; Too & Loakes n.d.; Gallego & Loakes 2007; Liang et al. 2012) can regenerate the blunt end irrespective of the primer sequence near its 3′ end. In one embodiment, regenerating the extendible end using degenerate or universal oligos can be accomplished by the following steps.

1. Incubate the primer with a degenerate (NNNNN) or universal complementary oligo,
2. Extend the primer with the desired nucleotide creating a 3′ overhang.
3. Denature the degenerate or universal complement by increasing temperature or other reversible chemical or physical means.
4. Reduce temperature or eliminate the denaturation conditions to allow the degenerate/universal complement to reposition itself and re-create the blunt end.
5. Go to step 2 for the next cycle.

If the universal or degenerate complement's melting temperature is reduced such that its duplex with the primer is not completely stable at reaction temperatures, the need for a denaturation step during synthesis will be eliminated.

The present disclosure contemplates alternative methods to regenerate the extendible end. For example, one can create conditions in which even a single-stranded DNA can interact with the enzyme active site similar to how double-stranded DNA would. For instance, attaching a degenerate or universal oligo directly to the polymerase and near its active site in such a way that it reconstitutes a blunt end for the enzyme at any point the enzyme binds to a free 3′ end, thus allowing extension by the following steps.

1. Incubate the enzyme-universal oligo complex with the primer,
2. Extend the primer with the desired nucleotide creating a 3′ overhang,
3. Denature the enzyme-universal oligo complex by increasing temperature or other reversible chemical or physical means,
4. Reduce temperature or eliminate the denaturation conditions to allow the enzyme-universal oligo complex to reposition itself to the newly generate 3′ end, and
5. Go to step 2 for the next cycle.

In some embodiments, the active site of the enzyme may be modified with other chemical moieties or non-standard amino acids to create conditions in which a 3′-end of a primer interacts with the active site in a similar fashion as a double-stranded blunt end, thus leading to template independent synthesis. For instance, a non-catalytic residue in or near the active site can be covalently attached to a short oligonucleotide in a way that upon the enzyme's interaction with the 3′ end of the primer, the attached oligo binds the primer, creating a double-stranded or double-stranded-like structure that would trigger the terminal transferase activity in perpetuity. In one embodiment, the short oligo could be attached to the protein from its 5′ end, in another embodiment it could be attached from its 3′ end. In another embodiment the oligo may be degenerate or be constituted, fully or partially, of universal bases. Another instance would be an enzyme with a non-standard amino-acid in its active site that upon interaction with the primer's 3′ end would create a structure similar to double-stranded DNA and thus trigger terminal transferase activity. In one embodiment, such a non-standard amino acid can have an organic base side chain such as adenine, guanine, cytosine, uracil, thymine, xanthine, hypoxanthine. In another embodiment, such a non-standard amino acid can have a single nucleoside side chain such as adenosine, guanosine, cytidine, uridine, thymidine, xanthosine, or inosine. In another embodiment, the side-chain could be a polynucleotide comprised of adenosine, guanosine, cytidine, uridine, thymidine, xanthosine, or inosine.

The present disclosure contemplates enhancing the accuracy of DNA synthesis by incorporating reversible terminator nucleotide analogues. Unlike TdT which does not efficiently use reversible-terminator nucleotide analogues as a substrate, polymerases such as exo-minus Klenow and Therminator are coveted for their ability to add various modified nucleotides with ease (Chiaramonte et al. 2003; Kincaid 2005; Franke-Whittle et al. 2006; Brakmann 2004).

The following examples are set forth as being representative of the present disclosure. These examples are not to be construed as limiting the scope of the present disclosure as these and other equivalent embodiments will be apparent in view of the present disclosure, figures and accompanying claims.

### Example 1

Single-Base Terminal Transferase Assay for Nine DNA Polvmerases and dCTP with and without Manganese.
1—Terminal transferase reactions were assembled to have the following composition for each sample:
Water: 7.5 μl
5× PolBuffer: 4 μl
25 uM Primer: 1 μl
S100 uM Complement: 0.5 μl
50 mM MgCl₂: 2 μl
20 mM MnCl₂ or water: 2 μl
heating to 80° C. for 1 min followed by cool down to room temperature
Enzyme: 1 μl
10 mM dCTP or water: 2 μl

5× PolBuffer: 100 mM Tris.HCl pH=8.0, 250 mM KCl

Primer:
(SEQ ID NO: 3)
AGATCAATTAATACGATACCTGCG
Complement:
(SEQ ID NO: 4)
CGCAGGTATC TTTTT/3InvdT/

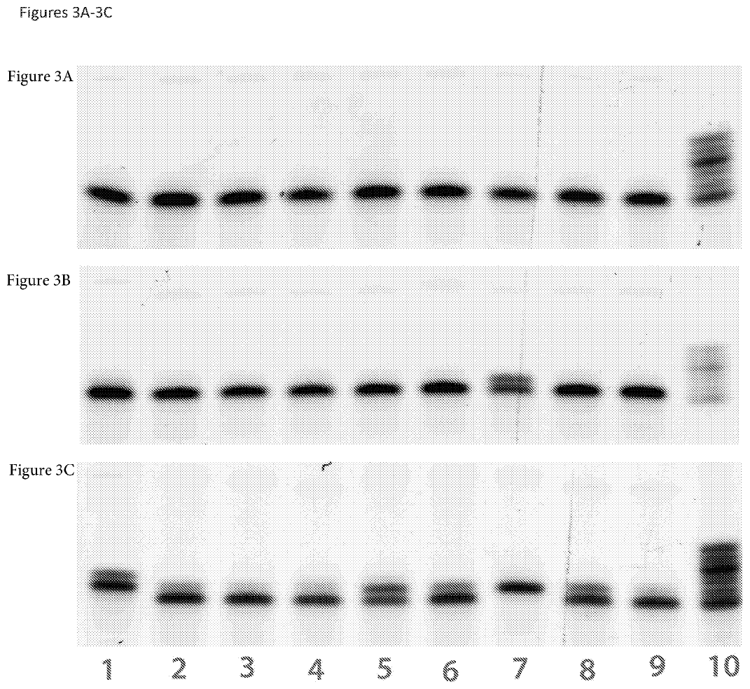

Enzyme: Bst, full length; or Klenow, Exo-; or Bsu. Large Fragment; or Sulfolobus; or Taq; or Therminator; Deep Vent, Exo-; or OmniAmp (PyroPhage exo-); or Vent, Exo.
   2—Reactions were incubated at 37° C. (for 10 minutes.
   3—Reactions were stopped by receiving 10 μl STOP&LOAD (2×Novex Urea Sample Buffer with 10 mM EDTA).
   4—Mixtures were heated to 80° C. for 3 min and cooled down on ice.
   5—5 ul of each loading mix was loaded on a 15% TBE-Urea gel. Gel was run in IX TBE at 180V for 90 minutes.
   6—After running, the gel was stained in 1× SybrGold in IX TBE for 15 minutes, rinsed once with 1×TBE, and imaged on the GelDoc with 5 second exposure in the SybrGold channel (FIG. 3A-3C).

Results for each enzyme without dCTP or manganese (FIG. 3A), with dCTP and without manganese (FIG. 3B), and with dCTP and manganese (FIG. 3C) are shown. They show that only Therminator has a slight terminal transferase activity with dCTP without manganese, whereas all enzymes have at least 10% activity with manganese and Klenow and Therminator have complete activities with manganese.

### Example 2

Multi-Cycle Extension of a Primer with a Degenerate Complementary Oligonucleotide.
1—Four extension reactions were assembled to have the following composition:
5× PolBuffer: 2 μl
1 uM Primer: 1 μl
250 uM Complement: 5 μl
100 mM MgCl₂: 0.5 μl
40 mM MnCl₂: 0.5 μl
heating to 80 C for 1 min followed by cool down to room temperature
Therminator: 0.5 μl
10 mM dATP: 0.5 μl

5× PolBuffer: 100 mM Tris.HCl pH=8.0, 250 mM KCl

Primer:
(SEQ ID NO: 3)
AGATCAATTAATACGATACCTGCG
Complement:
NNN NNN N/3InvdT/

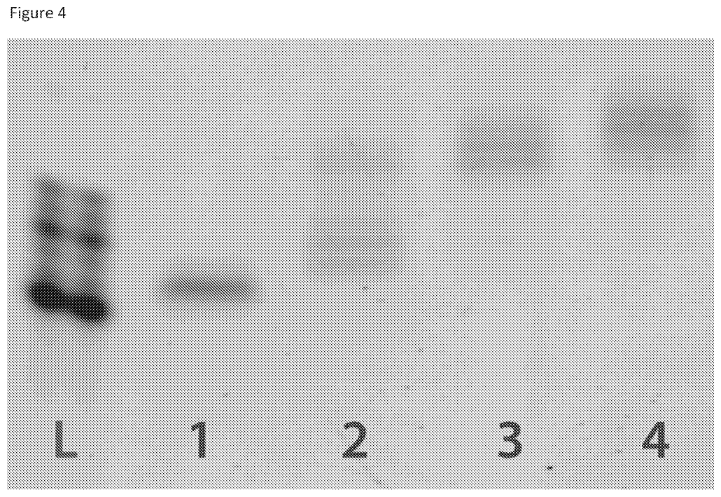

2—Reactions were placed in thermal cycler.
3—The thermal cycler was programmed such that each cycle would consist of: 1 minute at 25° C. 1 minute at 37° C., 1 minute at 75° C.
4—The four reactions were subjected to 1, 2, 3, or 5 rounds of thermal cycling.
5. Each reaction was mixed with 10 μl STOP&LOAD (2×Novex Urea Sample Buffer+10 mM EDTA).
6—Mixtures were heated to 85° C. for 3 min and cooled down on ice.
7—8 ul of each loading mix was loaded on a 15% TBE-Urea gel. Gel was run in 1×TBE at 180V for 85 minutes.
8—After running, the gel was stained in 1× SybrGold in 1×TBE for 15 minutes, rinsed once with 1×TBE, and imaged on the GelDoc with 5 second exposure in the SybrGold channel (FIG. 4).

The results show that while after 1 extension cycle the primer has only been extended by one nucleotide, after two cycles it is extended by 2 to 8 nucleotides, after three cycles by 4 to 10 nucleotides, and after 5 cycles by 7 to 12 nucleotides. This observation of multiple rounds of extension suggests that the blunt end was reconstituted after denaturation of the degenerate complement at 75° C. followed by re-annealing at 25° C.

### REFERENCES

Brakmann, S., 2004. High-density labeling of DNA for single molecule sequencing. Methods in molecular biology, 283, pp. 137-144.
Chang, L. M. & Bollum, F. J., 1986. Molecular biology of terminal transferase. CRC critical reviews in biochemistry, 21(1), pp. 27-52.
Chiaramonte, M. et al., 2003. Facile Polymerization of dNTPs Bearing Unnatural Base Analogues by DNA Polymerase α and Klenow Fragment (DNA Polymerase I)t. Biochemistry, 42(35), pp. 10472-10481.
Clark, J. M., 1988. Novel non-templated nucleotide addition reactions catalyzed by procaryotic and eucaryotic DNA polymerases. Nucleic acids research, 16(20), pp. 9677-9686.
   Clark, J. M., Joyce, C. M. & Beardsley, G. P., 1987. Novel blunt-end addition reactions catalyzed by DNA polymerase I of Escherichia coli. Journal of molecular biology, 198(1), pp. 123-127.
Delarue. M. et al., 2002. Crystal structures of a template-independent DNA polymerase; murine terminal deoxynucleotidyltransferase. The EMBO journal, 21(3), pp. 427-439.
Deng, G. & Wu. R., 1981. An improved procedure for utilizing terminal transferase to add homopolymers to the 3′ termini of DNA. Nucleic acids research, 9(16). pp. 4173-4188.
Derbyshire, V. et al., 1988. Genetic and crystallographic studies of the 3′,5′-exonucleolytic site of DNA polymerase I. Science, 240(4849), pp. 199-201.
Franke-Whittle, I. H. et al., 2006. Comparison of different labeling methods for the production of labeled target DNA for microarray hybridization. Journal of microbiological methods, 65(1), pp. 117-126.
Gallego, J. & Loakes. D., 2007. Solution structure and dynamics of DNA duplexes containing the universal base analogues 5-nitroindole and 5-nitroindole 3-carboxamide. Nucleic acids research, 35(9), pp. 2904-2912.
Kincaid, K., 2005. Exploration of factors driving incorporation of unnatural dNTPS into DNA by Klenow fragment (DNA polymerase I) and DNA polymerase. Nucleic acids research, 33(8), pp. 2620-2628.
Liang, F., Lindsay, S. & Zhang, P., 2012. 1,8-Naphthyridine-2,7-diamine: a potential universal reader of Watson-Crick base pairs for DNA sequencing by electron tunneling. Organic & biomolecular chemistry, 10(43), p. 8654.
Liang, F., Liu, Y.-Z. & Zhang. P., 2013. Universal base analogues and their applications in DNA sequencing technology. RSC advances, 3(35), p. 14910.
Loakes, D., 2001. SURVEY AND SUMMARY: The applications of universal DNA base analogues. Nucleic acids research, 29(12). pp. 2437-2447.
Mead. D. et al., Novel Tailing Enzymes for Next Gen Library Construction, http://www.lucigen.com/. Available at: http://www.lucigen.com/docs/posters/Novel-Tailing-Enzymes-for-Next-Gen-Library-Construction.pdf [Accessed Apr. 7, 2017].
Mir, K. U. et al., 2009. Sequencing by Cyclic Ligation and Cleavage (CycLiC) directly on a microarray captured template. Nucleic acids research, 37(1), p.e5.
Miyaki, M. et al., 1977. Effect of metal cations on misincorporation by E. coli DNA polymerases. Biochemical and biophysical research communications, 77(3). pp. 854-860.
Mole. S. E., Iggo. R. D. & Lane. D. P., 1989. Using the polymerase chain reaction to modify expression plasmids for epitope mapping. Nucleic acids research, 17(8), p. 3319.
Motea. E. A. & Berdis. A. J., 2010. Terminal deoxynucleotidyl transferase: the story of a misguided DNA polymerase. Biochimica et biophysica acta, 1804(5), pp. 1151-1166.
Strauss, B. S., 2002. The “A” rule revisited: polymerases as determinants of mutational specificity. DNA repair, 1(2), pp. 125-135.
Too, K. & Loakes. D., Universal Base Analogues and their Applications to Biotechnology. In Modified Nucleosides. pp. 277-303.
Yang, S.-W., 2002. Mutant Thermotoga neapolitana DNA polymerase I: altered catalytic properties for non-templated nucleotide addition and incorporation of correct nucleotides. Nucleic acids research, 30(19), pp. 4314-4320.

### Other Embodiments

Other embodiments will be evident to those of skill in the art. It should be understood that the foregoing description is provided for clarity only and is merely exemplary. The spirit and scope of the present invention are not limited to the above examples, but are encompassed by the following claims. All publications and patent applications cited above are incorporated by reference in their entirety for all purposes to the same extent as if each individual publication or patent application were specifically and individually indicated to be so incorporated by reference.

1. A method for adding one or more selected nucleotides to an extendible end of a double stranded oligonucleotide initiator comprising
(a) providing a first single stranded oligonucleotide
(b) providing a second single stranded oligonucleotide under conditions wherein the first single stranded oligonucleotide anneals to the second single stranded oligonucleotide thereby forming the double stranded oligonucleotide initiator having an extendible end comprising a 3′ terminal nucleotide of the first single stranded oligonucleotide,
(c) providing a reaction mixture to the double stranded initiator wherein the reaction mixture comprises a template-dependent DNA polymerase, one or more selected nucleotide triphosphates, and divalent cations, and wherein the template-dependent DNA polymerase adds one or more of the selected nucleotide triphosphates to the 3′ terminal nucleotide of the first single stranded oligonucleotide of the extendible end of the double stranded oligonucleotide initiator.

2. The method of claim 1 wherein 3′ end terminal nucleotide of the second single stranded oligonucleotide is inactivated from extension.

3. The method of claim 2 wherein the 3′ end terminal nucleotide of the second single stranded oligonucleotide lacks a 3′ hydroxyl group for extension.

4. The method of claim 1 wherein the extendible end is extended by a template-dependent DNA polymerase via its terminal transferase activity.

5. The method of claim 1 wherein the extendible end comprises a blunt end, a 5′ overhang, a short 3′ overhang, a mixture thereof, or an equilibrium mixture thereof.

6. The method of claim 1 wherein the template-dependent DNA polymerase has terminal transferase activity.

7. The method of claim 6 wherein the template-dependent DNA polymerase lacks 3′ to 5′ proofreading activity.

8. The method of claim 6 wherein the template-dependent DNA polymerase comprises Bst, Klenow Exo-, Bsu, Sulfolobus, Taq, Therminator, Deep Vent Exo-, OmniAmp, Vent Exo-, Phi29 Exo-, T4 DNA polymerase Exo-, T7 DNA polymerase Exo-, Tth polymerase, Pfu Exo-, E. coli DNA Polymerase I Exo-, 9° N™ DNA polymerase, Pwo Exo-, Pab Exo-, and the like.

9. The method of claim 6 wherein the template-dependent DNA polymerase having terminal transferase activity is mutated or otherwise engineered to have reduced or abrogated template-dependent activity.

10. (canceled)

11. The method of claim 1, wherein the nucleotide triphosphate comprises a base-modified nucleotide analogue, a sugar-modified nucleotide analogue, or a triphosphate-modified nucleotide analogue.

12.-14. (canceled)

15. The method of claim 1 wherein the nucleotide triphosphate comprises dATP, dTTP, dCTP, dGTP, or dUTP.

16. The method of claim 6 wherein the terminal transferase activity of the template-dependent polymerase is modulated by presence of non-magnesium divalent cations.

17. The method of claim 6 wherein the terminal transferase activity of the template-dependent polymerase is modulated by the presence of manganese, cobalt, zinc, or nickel.

18.-21. (canceled)

22. The method of claim 16 wherein the terminal transferase activity of the template-dependent polymerase is modulated by presence of non-magnesium divalent cations and wherein the template-dependent polymerase adds nucleotide triphosphates to the extendible end comprising a blunt end, a 5′ overhang, a short 3′ overhang, a mixture thereof, or an equilibrium mixture thereof with enhanced activity.

23.-29. (canceled)

30. The method of claim 1 where the divalent cations comprise one or more of magnesium, manganese, cobalt, nickel, zinc, cadmium, or calcium.

31. A method for enhancing terminal-transferase activity of a template-dependent polymerase comprising supplementing an effective amount of non-magnesium divalent cations to a reaction mixture wherein the reaction mixture comprises
i) buffer, salt, and the template-dependent DNA polymerase having terminal-transferase activity,
ii) a double stranded oligonucleotide initiator having an extendible end,
iii) a selected set of nucleotide triphosphates, and
iv) divalent cations,
wherein the double stranded oligonucleotide initiator is formed by annealing a first single stranded oligonucleotide to a second single stranded oligonucleotide, and wherein the 3′ terminal nucleotide of the first single stranded oligonucleotide of the extendible end of the double stranded oligonucleotide initiator is extended by the terminal transferase activity of the template-dependent DNA polymerase in a template independent fashion.

32.-113. (canceled)

114. A method for making a polynucleotide comprising
(a) providing a first single stranded oligonucleotide,
(b) providing a degenerate or universal single stranded oligonucleotide under conditions wherein the first single stranded oligonucleotide anneals to the degenerate or universal single stranded oligonucleotide thereby forming a double stranded oligonucleotide initiator having an extendible end comprising the 3′ terminal nucleotide of the first single stranded oligonucleotide,
(c) providing a reaction mixture to the double stranded initiator wherein the reaction mixture comprises an enzyme, a selected nucleotide triphosphate, and divalent cations, and wherein the enzyme extends the extendible end,
(d) regenerating an extendible end of the extended template, and
(e) repeating steps (c) to (d) until a polynucleotide of a desired sequence or information content is formed, with the proviso that step (d) is not required to be performed after the polynucleotide is formed.

115.-193. (canceled)
